(19) United States
(12) Patent Application Publication
(10) Pub. No.: US 2016/0354070 A1
(43) Pub. Date: Dec. 8, 2016

(54) TREATMENT METHOD FOR HOLLOW ORGANS

(71) Applicant: OLYMPUS CORPORATION, Tokyo (JP)

(72) Inventor: Kosuke MOTAI, Hidaka-shi (JP)

(21) Appl. No.: 14/733,636

(22) Filed: Jun. 8, 2015

(51) Int. Cl. A61B 17/00; A61B 18/14

(52) U.S. Cl. CPC A61B 17/00234; A61B 18/1477; A61B 2017/00269; A61B 2017/00278; A61B 2017/00296; A61B 2017/00358; A61B 2017/00818; A61B 2018/00601

(57) ABSTRACT

A tissue treatment method combined with a bypass procedure of a hollow organ includes a first process of dividing the hollow organ into a first region of a distal side of the hollow organ and a second region of a proximal side of the hollow organ, a second process of forming a damaged part of a predetermined size on an outer surface of at least one of the first and second regions, and a third process of fixing the first and second regions such that the damaged part is kept in close contact with an outer surface of the other of the first and second regions.

### BACKGROUND OF THE INVENTION

Field of the Invention

The present invention relates to a treatment method for hollow organs.

Description of Related Art

Conventionally, as a surgical procedure for obesity, Roux-en-Y gastric bypass has been known (e.g. see US2011/0113863A1). In a Roux-en-Y gastric bypass for obesity, the stomach is divided into one region (a pouch) connected to the esophagus and another region (the remaining stomach) connected to the duodenum. Afterward, the jejunum is cut, a cut end of a distal side is anastomosed to the pouch, and the cut end of a proximal side is anastomosed to the distal side of the jejunum anastomosed with the pouch.

Due to this procedure, as food ingested from the mouth passes only the pouch in which the food can be stored only in small amounts, ingestion of excessive food is suppressed. Also, since a part of the jejunum is bypassed and is not used for digestion and absorption, the ability to digest and absorb the ingested food is also reduced compared to prior to the procedure. Due to these behaviors, it can improve obesity.

### SUMMARY OF THE INVENTION

The present invention provides a tissue treatment method combined with a bypass procedure of a luminal organ, which includes a first process of dividing the hollow organ into a first region of a distal side of the hollow organ and a second region of a proximal side of the hollow organ, a second process of forming a damaged part of a predetermined size on an outer surface of at least one of the first and second regions, and a third process of fixing the first and second regions such that the damaged part is kept in close contact with an outer surface of the other of the first and second regions.

### DESCRIPTION OF THE DRAWINGS

FIG. 1 is a view illustrating a first process in a tissue treatment method according to an embodiment of the present invention.

FIG. 2 is a view illustrating a second process of the tissue treatment method.

FIG. 3 is a view illustrating a third process of the tissue treatment method.

FIG. 4 is a view illustrating a pouch and a remaining stomach coapted by adhesion wherein the pouch and the remaining stomach including partially cutaway view.

FIG. 5 is a view illustrating an operation of specifying an adhered part after a bypass procedure.

FIG. 6 is a view illustrating another example of the operation of specifying the adhered part.

FIG. 7 is a view illustrating a state in which an optical endoscope is introduced into a remaining stomach from a through-hole.

FIG. 8 is a view illustrating an operation of closing the through-hole.

FIG. 9 is a view illustrating an example of the treatment tool for forming the damaged part and a damaged part formed by the same treatment tool.

FIG. 10A is a view illustrating an example of an operation of forming the damaged part.

FIG. 10B is a view illustrating the example of the operation of forming the damaged part.

FIG. 11 is a view illustrating an example of the treatment tool for forming the damaged part and a damaged part formed by the same treatment tool.

FIG. 12 is a view illustrating an example of the treatment tool for forming the damaged part and a damaged pan formed by the same treatment tool.

FIG. 13 is a view illustrating an example of the treatment tool for forming the damaged part and a damaged part formed by the same treatment tool.

FIG. 14 is a view illustrating another example of first and second regions.

FIG. 15 is a view illustrating another example of first and second regions.

FIG. 16 is a view illustrating another example of first and second regions.

FIG. 17 is a view illustrating another aspect of the third process.

### DETAILED DESCRIPTION OF THE INVENTION

One problem of a Roux-en-Y gastric bypass is an endoscopic approach to a duodenum, a bile duct, a remaining stomach, etc. after a procedure. Since a stomach is divided into a pouch and a remaining stomach, a distal end of an endoscope inserted from a mouth is first advanced to an anastomotic part with a jejunum in order to approach each of the aforementioned pieces of tissue using the endoscope, and the endoscope should be advanced so as to retrograde from there toward the duodenum. This approach has a very high level of difficulty because a path is very long and a manipulating strength of the endoscope is not easily transmitted to the distal end of the endoscope in the retrograde approach. For this reason, in patients who have received a Roux-en-Y gastric bypass, there is often no alternative but to give up the endoscopic approach to each of the aforementioned pieces of tissue.

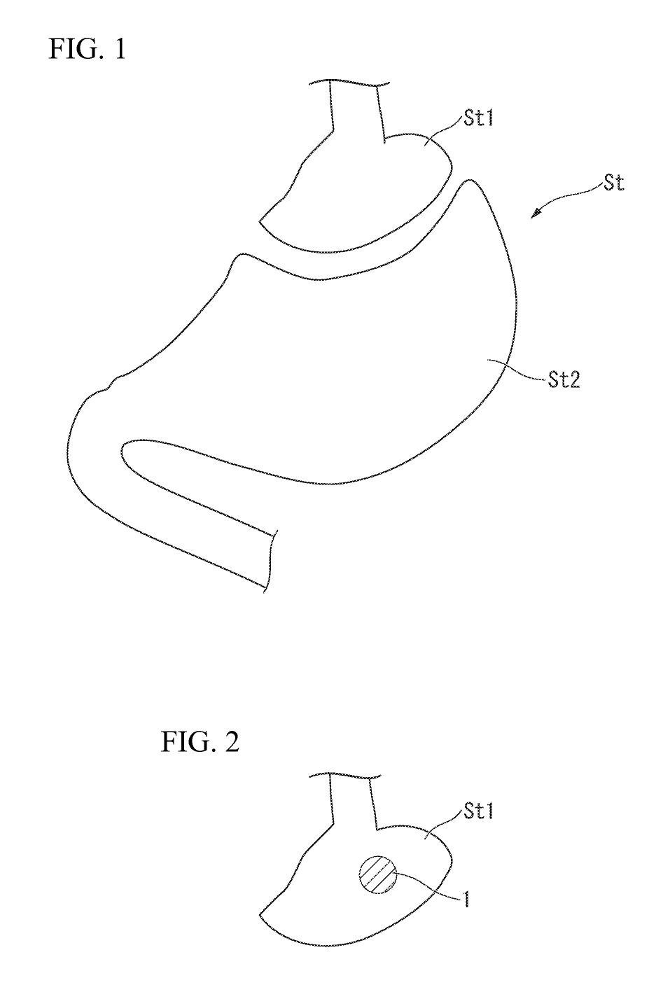

In the present invention, in consideration of the above problems, predetermined processes are added to the bypass procedure of the hollow organs, and thereby an approach to predetermined tissue after the procedure is remarkably facilitated. Here, an embodiment of the present invention will be described with reference to FIGS. 1 to 12.

First, as illustrated in FIG. 1, an operator divides a stomach St into a pouch (first region) St1 of a distal side of the stomach St and a remaining stomach (second region) St2 of a proximal side of stomach St (first process). The first process may be performed in the same way as the known Roux-en-Y gastric bypass. For example, the first process may be performed using a medical stapler. Sizes (volumes) of the pouch St1 and the remaining stomach St2 may be adequately set in consideration of conditions of a patient, and so on.

Next, as illustrated in FIG. 2, the operator artificially forms a damaged part 1 having a predetermined size on an outer surface (surface exposed to an abdominal cavity) of the pouch St1 (second process).

In the present invention, the term “damage” refers to the extent to which self-repair of tissue so at to generate adhesion to be described below is initiated in an injury that does not communicate with a lumen of the hollow organ. Accordingly, as long as the damage is sufficient to initiate self-repair, its type or form such as excoriation, rupture, burn, loss of tissue, a chemical injury, etc., does not matter.

The size of the damaged part 1 can be adequately set. However, since there is a possibility of passing the endoscope after the procedure as will be described below. As the damaged part 1, for instance, a circle having a diameter of 20 mm or more is preferred. As long as the size of the damaged part is a circle having a diameter of 20 mm or more, it is possible to form a through-hole into which almost the entirety of the endoscope currently being used can be inserted in the order to be described below. When the damaged part is not a circle, a form in which a circle inscribed within the damaged part has a diameter of 20 mm or more is preferred.

The damaged part may not be formed in a sheet shape. For example, the damaged part may be formed in a line or strip shape surrounding a predetermined region. In this case, the size of the surrounded region is defined as the size of the damaged part. Therefore, the surrounded region preferably has dimensions meeting the aforementioned conditions.

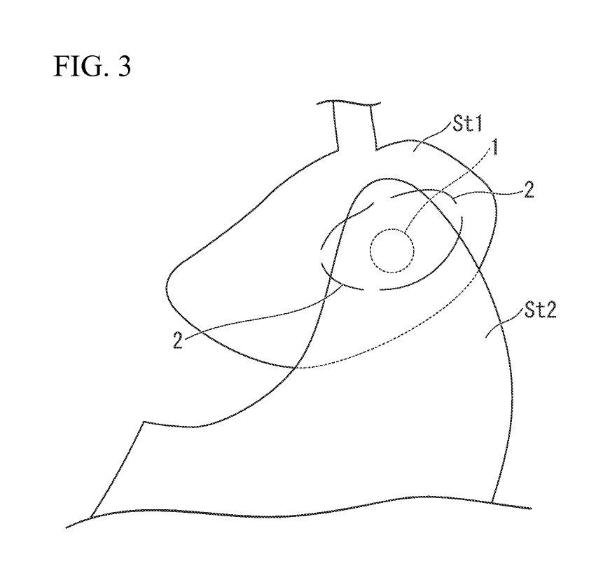

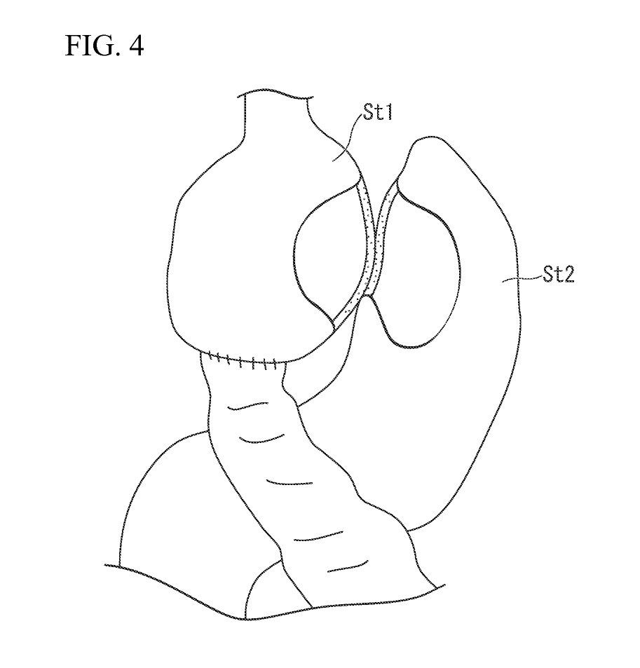

Next, as illustrated in FIG. 3, in a state in which an outer surface of the remaining stomach St2 is in close contact with the damaged part 1, the remaining stomach St2 is fixed to the pouch St1 using a suture 2, and a close contact state therebetween is maintained (third process).

The tissue treatment method of the present embodiment is completed through the aforementioned first to third processes. The operator performs the remaining processes of the Roux-en-Y gastric bypass as needed, and may perform the tissue treatment method of the present embodiment after the procedure of the Roux-en-Y gastric bypass is completed. Further, one or more processes of the Roux-en-Y gastric bypass may be performed between processes of the tissue treatment method.

After the procedure, the self-repair of the damaged part 1 is initiated in the body of the patient. Since the damaged part 1 is in close contact with the remaining stomach St2, the damaged part 1 and the remaining stomach St2 are adhered in a process in which the damaged part 1 is self-repaired, and thereby the pouch St1 and the remaining stomach St2 are coapted.

A mechanism of the adhesion will not be fully explained, but a known mechanism includes inter-tissue adhesion using collagen. Among polymers contained in body tissue, collagen is one of the materials having highest adhesiveness. Therefore, an injury in which a protein containing collagen is exposed is a preferred form of the damage to the damaged part of the present invention.

The pouch St1 and the remaining stomach St2 are adhered by coapting them in a non-communicating state as illustrated in FIG. 4, and thus the remaining stomach St2 does not separate from the pouch St1 after the procedure. Accordingly, when there is a need to approach the remaining stomach St2 or the duodenum using the endoscope after the procedure for any reason, a hole is made in a part coapted by the adhesion, and thereby the endoscope can be easily introduced into the remaining stomach St2 or the duodenum.

An order in which the remaining stomach St2 or the duodenum is accessed after the procedure will be described.

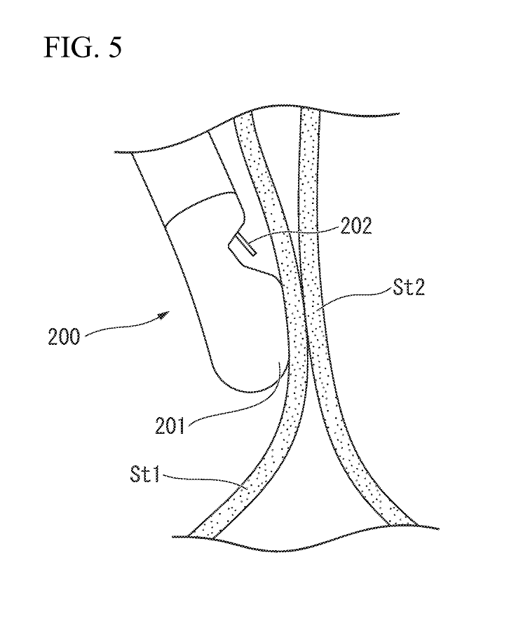

First, as illustrated in FIG. 5, an operator introduces an ultrasonic endoscope 200 into the pouch St1, and specifies a part at which the pouch St1 and the remaining stomach St2 are adhered while pressing an ultrasonic probe 201 against a wall of the pouch St1. The adhered part may be specified by verifying that the wall of the pouch St1 and a wall of the remaining stomach St2 are coapted into one and that there is a wide space located ahead by an ultrasonic tomographic image.

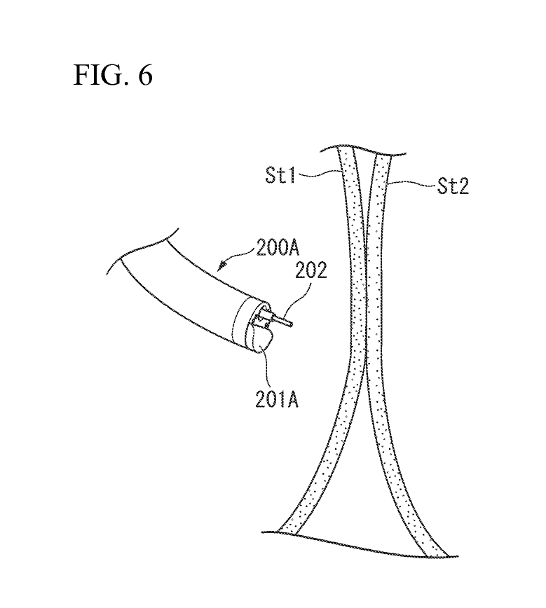

After the adhered part is specified, the ultrasonic endoscope 200 is removed and replaced with an optically observable endoscope such as a video scope. Prior to the removal of the ultrasonic endoscope 200, as illustrated in FIG. 5, a high-frequency knife 202 may be projected from a channel of the ultrasonic endoscope 200, and cauterized parts having spot shapes may be formed on the wall of the pouch St1 in a desired number and used as a guide for the adhered part. At this time, as illustrated in FIG. 6, an ultrasonic endoscope 200A of a type in which the ultrasonic probe 201A is not located on an extension line of the channel of the treatment tool may be used. In this case, a direction in which the ultrasonic endoscope 200A is advanced for pressing the ultrasonic probe 201A and a direction in which the high-frequency knife 202 is projected are matched with each other. Thus, there is an advantage that manipulation becomes easy.

After the optically observable endoscope (optical endoscope) is introduced into the pouch St1, the adhered part is incised using, for instance, the high-frequency knife inserted into the optical endoscope while the adhered part is being observed by the optical endoscope. Thereby, a though-hole 10 communicating the pouch St1 and the remaining stomach St2 with each other is formed (fourth process). Since the through-hole 10 is formed in the adhered part, the periphery of the through-hole 10 is sealed by adhesion. Therefore, a liquid, etc. inside the pouch St1 or the remaining stomach St2 does not leak from the through-hole 10 into the abdominal cavity.

When an endoscope capable of performing both ultrasonic observation and optical observation is used, after the adhered part is specified, the adhered part may be continuously incised to form the through-hole.

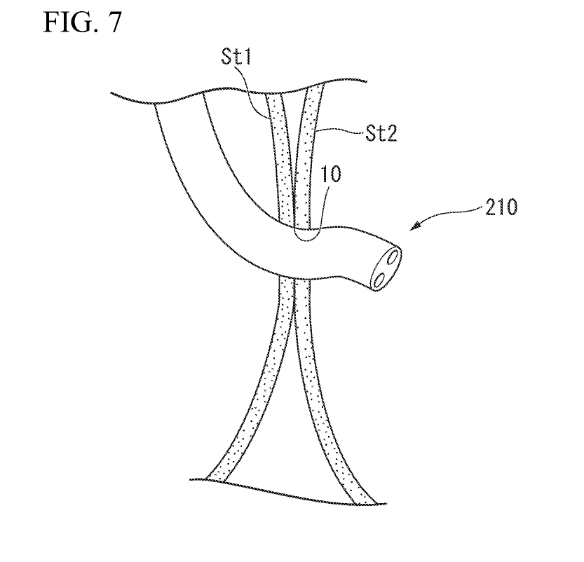

After the through-hole 10 is formed, the optical endoscope 210 can be introduced into the remaining stomach St2 by inserting the optical endoscope 210, as illustrated in FIG. 7 into the through-hole 10. When the optical endoscope 210 is introduced into the remaining stomach St2, a guide wire may be previously inserted into the through-hole 10, and the optical endoscope 210 may be inserted into the through-hole under guidance.

After the optical endoscope 210 is introduced into the remaining stomach St2, a desired procedure can be performed on the remaining stomach, the duodenum, the bile duct, and so on (fifth process). A specific example of the fifth process includes observation and lesion excision of the remaining stomach (including endoscopic mucosal resection (EMR) or endoscopic submucosal dissection (ESD)), observation and lesion excision of the duodenum (including polypectomy), incision of duodenal papilla, observation of the bile and pancreatic ducts including endoscopic retrograde cholangiopancreatography (ERCP), destruction and recovery of gallstones, placement of a bile duct stent, and so on. Each of the aforementioned procedures may be performed by a combination of the optical endoscope 210 and a scope or a treatment tool introduced into the abdominal cavity from, for instance, a trocar. This procedure is defined herein as “internal/external combined operation,” and also includes a case of performing a surgical operation while giving support from an alimentary canal. For example, when a lesion portion of the duodenum is excised using an instrument introduced into the abdominal cavity, an example in which the excision is also performed to give support from an endoscope introduced into the alimentary canal to optimize an excision margin is given.

The endoscope used in the fifth process is not limited to a direct-view endoscope illustrated in FIG. 7, and may be adequately selected from a variety of known endoscopes such as a side-view endoscope or an oblique-view endoscope depending on a target organ. Also, depending on specific contents of the fifth process, endoscopes other than the optical endoscope, such as an ultrasonic endoscope, may be used.

Since anterogradely approach can be performed on the remaining stomach, the duodenum, and the bile duct after the endoscope is introduced into the remaining stomach St2, the operator can perform a desired procedure in approximately the same order as under normal circumstances.

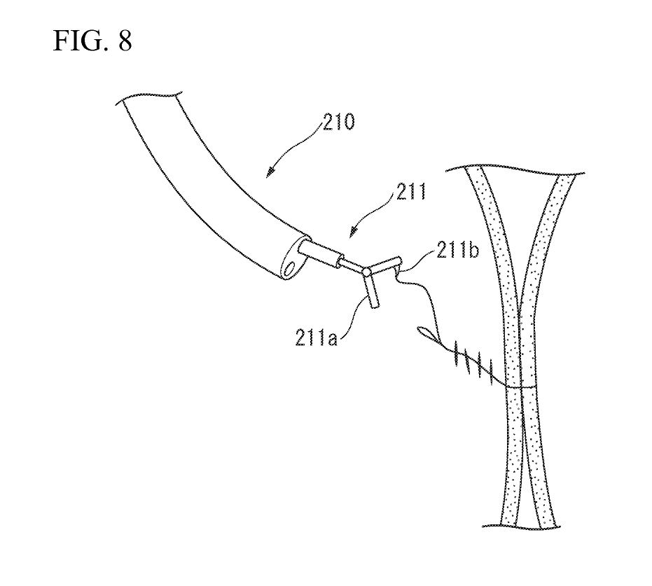

After the desired procedure is performed, the operator removes the optical endoscope 210 from the through-hole 10, and closes the through-hole 10 (sixth process). In FIG. 8, an example in which the through-hole 10 is closed by sutures using a suture instrument 211 that can be inserted into the optical endoscope 210 is illustrated. The known suture instrument 211 in which a needle member 211b locking a suture is delivered between a pair of jaws 211a is illustrated in FIG. 8. However, instead of the device, a variety of known devices such as a clamp using a suture unit in which an anchor is mounted on a suture or using a suture having a barb locked on tissue may be adequately selected.

As described above, according to the tissue treatment method of the present embodiment, in combination with the bypass procedure of the hollow organ, the path communicating the first region and the second region, which is separated in the non-communicating state, with each other can be easily provided after the bypass procedure. As a result, after the bypass procedure, the endoscope is possible to access desired spots easier compared to the approach through the lumen into which it is difficult to introduce the endoscope.

In the tissue treatment method of the present embodiment, various changes are possible.

In the second process, there is no particular limitation to means for forming the damaged part, which has already been described. Therefore, it is possible to use a variety of instruments for the surgical operation or a variety of energy treatment tools that treat tissue using a high-frequency current, heat, ultrasound, and so on. Hereinafter, an example of the treatment tool capable of easily forming the damaged part having a predetermined size is shown.

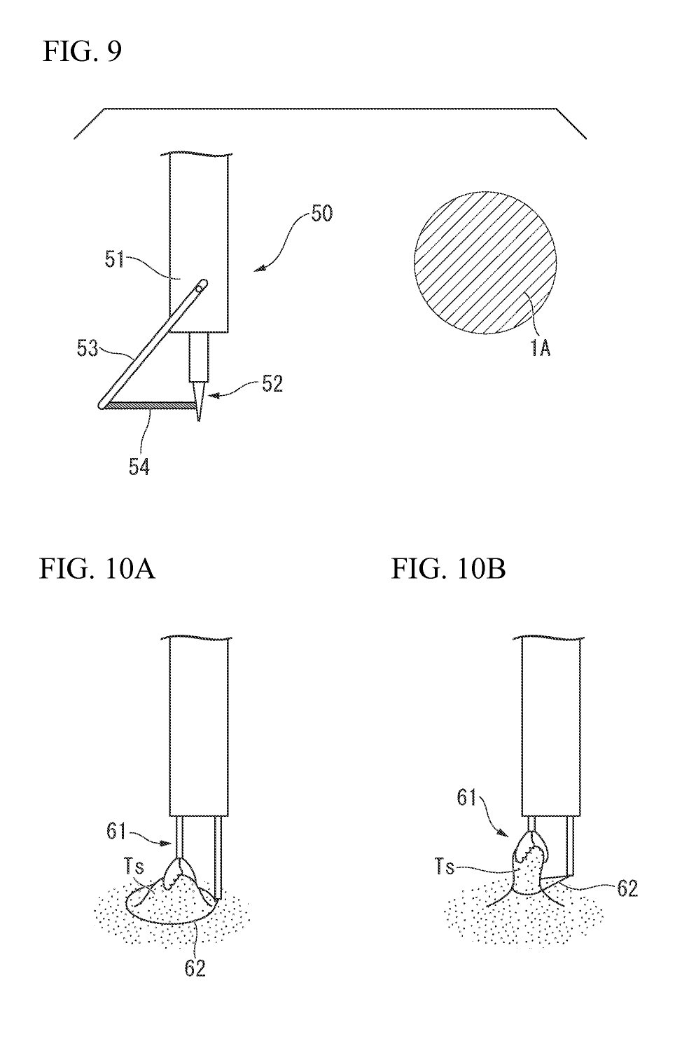

A treatment tool 50 illustrated on the left in FIG. 9 is provided with a needle part 52 which can protrude from or retract into a distal end of a sheath 51, an arm 53 which is rotatably mounted on the sheath 51, and a cauterizing part 54 which is provided between the arm 53 and the needle part 52. The treatment tool 50 has the same basic structure as a known endoscope. As a manipulating part (not shown) provided at a proximal end of the sheath 51 is manipulated, a protruding or retracting manipulation of the needle part 52, a rotating manipulation of the arm 53, and control of a current applied to the cauterizing part 54 are possible.

When the damaged part is formed using the treatment tool 50, the needle part 52 pierces the target tissue (the first region or the second region). Afterward, if the arm 53 is opened such that the distal end of the arm 53 is away from the needle part 52, and the treatment tool 50 is rotated about the needle part 52 one turn while a current is applied to the cauterizing part 54, a circular damaged part 1A illustrated on the right in FIG. 9 is formed.

In FIG. 10A, an example in which the damaged part is formed using gripping forceps 61 for a general endoscope and a snare wire 62 for the endoscope is illustrated. In a state in which the snare wire 62 is pressed against the target tissue, the tissue Ts surrounded by the snare wire 62 as illustrated in FIG. 10A is gripped and raised by the gripping forceps 61. While the operator keeps the tissue raised, if a base of the tissue is tied by the snare wire 62 as illustrated in FIG. 10B and is energized, the outer tissue is resected, and a damaged part is formed.

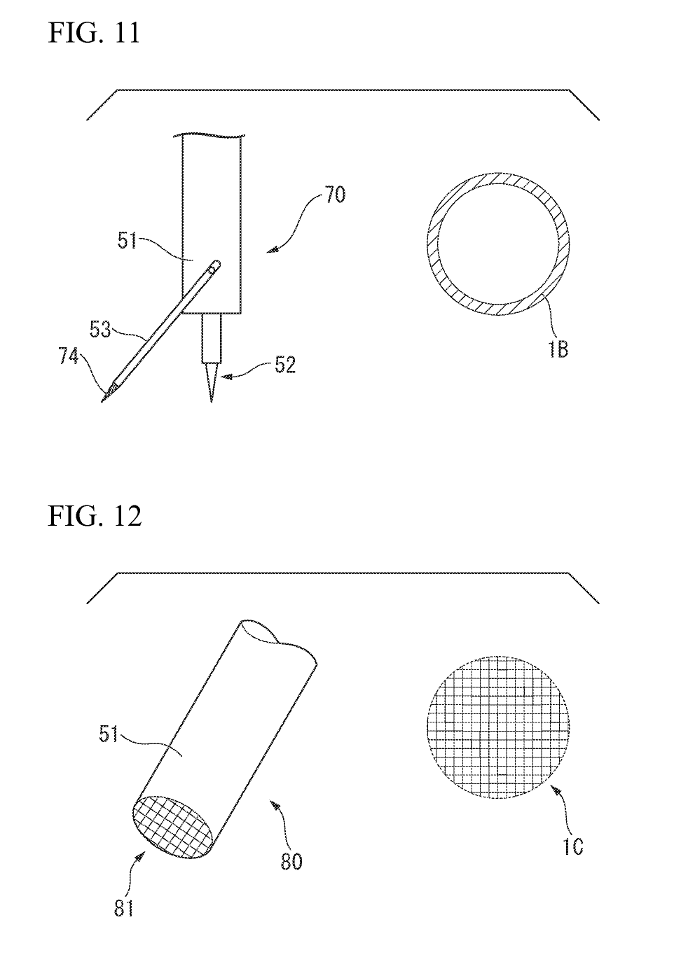

A treatment tool 70 illustrated on the left in FIG. 11 is provided with a needle part 52 and an arm 53 like the treatment tool 50. However, instead of the cauterizing part 54, a cauterizing part 74 is provided at a distal end of the arm 53. When a damaged part is formed using the treatment tool 70, the needle part 52 pierces the target tissue, and the arm 53 is opened and rotated one turn while a current is applied to the cauterizing part 74.

In the treatment tool 70, an annular damaged part 1B illustrated on the right in FIG. 1 is formed. When another region comes into close contact with the damaged part 1B in the third process, annular adhesion occurs according to the shape of the damaged part 1B. A first region and a second region are not adhered to each other at a portion surrounded by the annular adhered part. However, even if a through-hole 10 is formed inside the annular adhered part and an endoscope passes through the through-hole 10, since a periphery of the through-hole 10 is sealed by adhesion, the contents of a hollow organ do not leak into the abdominal cavity.

A treatment tool 80 illustrated on the left in FIG. 12 is provided with a grid-like cauterizing part 81 at a distal end of a sheath 51. When a damaged part is formed using the treatment tool 80, an energized cauterizing part 81 need only be pressed against tissue, and the treatment tool 80 need not be rotated. In the treatment tool 80, as illustrated on the right in FIG. 12, a grid shape damaged part 1C whose outer form is an approximately circular shape can be formed. When a form of a damaged part is like the damaged part 1C, the size of a shape connecting an outer edge of damage is defined as the size of the damaged part.

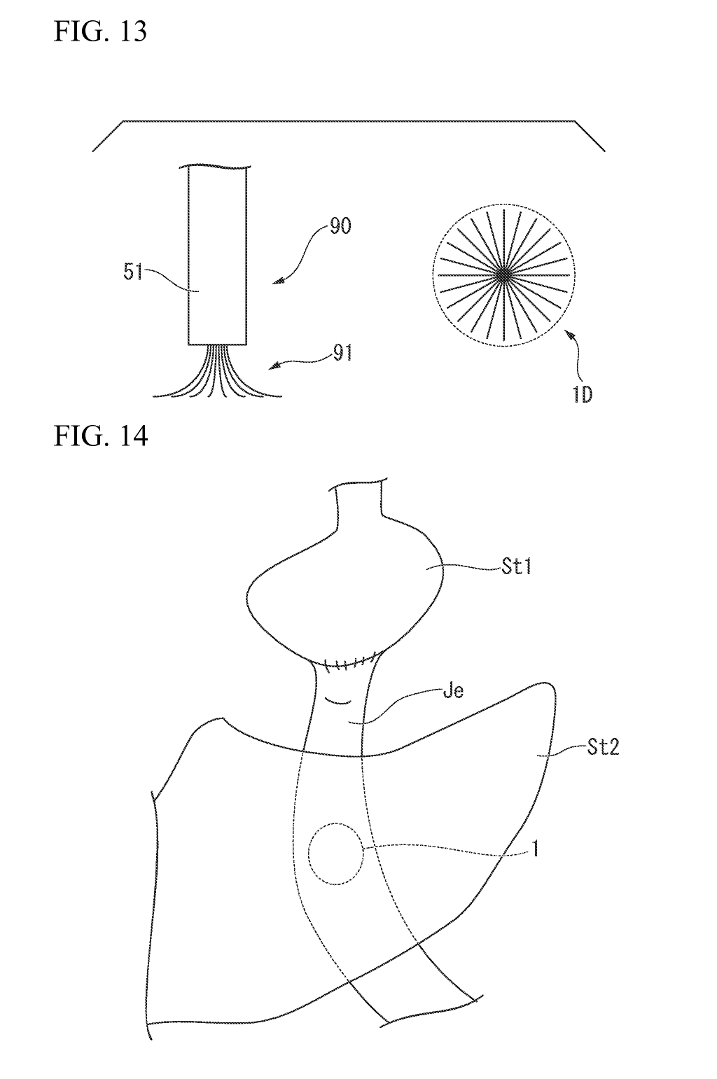

In a treatment tool 90 illustrated on the left in FIG. 13, a plurality of bundled cauterizing parts 91 are disposed so as to be able to protrude from or retract into a distal end of a sheath 51. The distal ends of the cauterizing parts 91 is configured to be changed their shape such that the ends spread out radially when protruding from the distal end of the sheath 51. When a damaged part is formed using the treatment tool 90, the energized cauterizing parts 91 need only be pressed against tissue, and the treatment tool 90 need not be rotated. In the treatment tool 90, as illustrated on the right in FIG. 13, a radial damaged part 1D can be formed. If the treatment tool 90 is slightly rotated about an axis of the sheath 51 while the energized cauterizing parts 91 are pressed against the tissue, it is also easy to form a circular damaged part.

In each of the aforementioned treatment tools, the cauterizing part may be an electrode supplied with a high-frequency current, or a heater energized to reach a high temperature.

Although one embodiment of the present invention has been described, the technical scope of the present invention is not limited to the aforementioned embodiment, but it is possible to change a combination of the components, and apply or remove various modifications to or from each component without departing from the spirit or teachings of the present invention.

For example, in the second process of the present invention, the damaged part may be formed at only one or both of the pieces of tissue generating the adhesion as described above. When the damaged part is formed at only one of the pieces of tissue, the damaged part may be formed at any tissue. Therefore, in the aforementioned example, the damaged part may be formed only at the remaining stomach St2. When the damaged part is formed at both of the pieces of tissue, the forms of the respective damaged parts may be the same as or different from each other.

Further, the tissue generating the adhesion is not limited to the aforementioned pouch and remaining stomach.

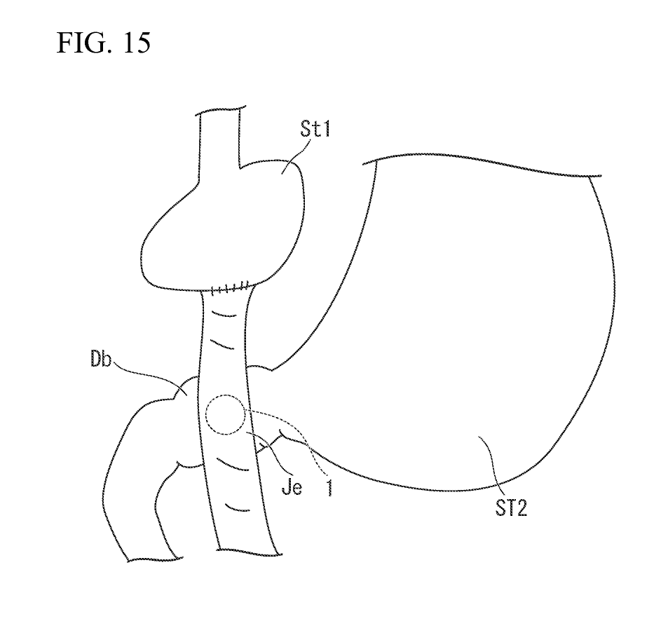

For example, as illustrated in FIG. 14, the jejunum (second region) Je anastomosed with the pouch St1 and the remaining stomach (first region) St2 may be adhered. As illustrated in FIG. 15, the jejunum Je anastomosed with the pouch St1 and the duodenal bulb (first region) Db connected to the remaining stomach St2 may be adhered.

However, if one of the first and second regions being coapted by the adhesion is used as the remaining stomach, the endoscope protrudes into the remaining stomach having a relatively wide space when the endoscope is inserted into the formed through-hole. Thus, there are merits in that subsequent manipulation becomes easy and that the endoscope can anterogradely approach both the remaining stomach and the duodenum.

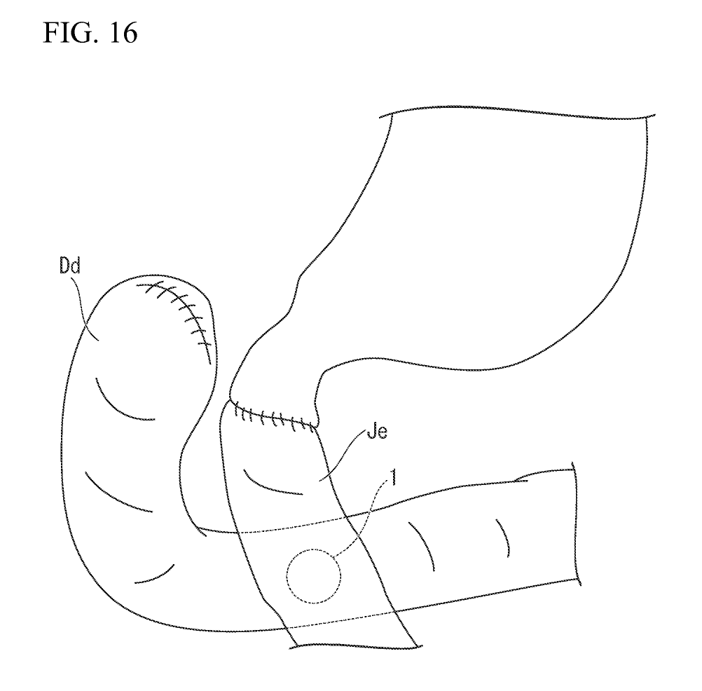

Also, the surgical procedure to which the tissue treatment method of the present invention is applied is not limited to the aforementioned Roux-en-Y gastric bypass. In FIG. 16, an example of the tissue treatment method of the present invention is applied to a so-called duodenal switch in which a pyloric part and a duodenum are divided, a distal side of a jejunum is anastomosed to the pyloric part is illustrated. Instead of an example in which a duodenum (first region) Dd and a jejunum Je are adhered which illustrated in FIG. 16, a stomach (first region) and a duodenum (second region) may be adhered. In addition, the tissue treatment method of the present invention may also be applied to gastric cancer resection based on the Roux-en-Y gastric bypass.

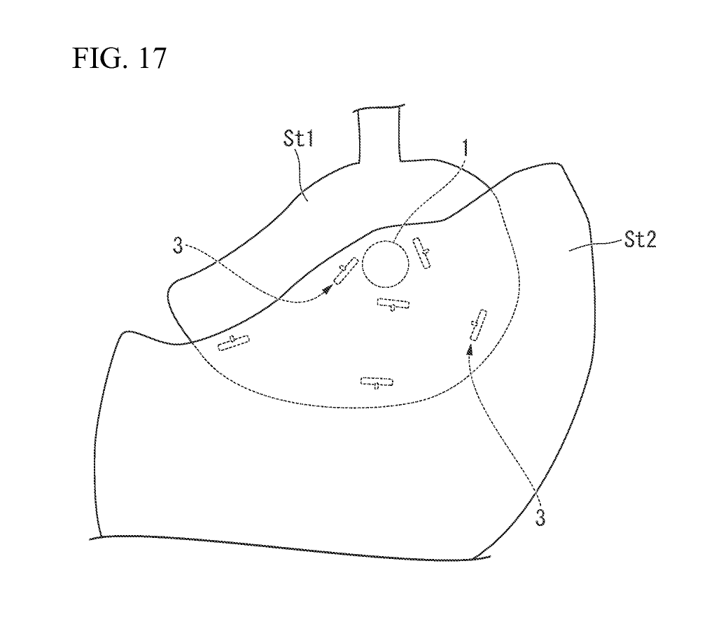

In the third process, the means for fixing the second region is not limited to the aforementioned suture. Therefore, the means may be a tagged suture unit 3 as illustrated in FIG. 17. As long as the second region can be reliably fixed until the adhesion occurs, the second region may be temporarily fixed using a suture made of a biodegradable material.

Here, a portion at which the second region is fixed is not limited to the periphery of the damaged part. Therefore, as illustrated in FIG. 17, a plurality of spots spaced apart from the damaged part 1 may be fixed. Thereby, for example, when the remaining stomach St2 is remarkably greater than the pouch St1, a situation in which a load is concentrated on the adhered damaged part and the adhered damaged part is separated can be suitably prevented. When the plurality of spots apart from the damaged part are fixed, damage may be formed independently of the damaged pan, and fixation may be performed by adhesion.

Also, in the second process, marking may be performed on an inner wall around the portion at which the damaged part is formed, for instance, by a clip. Since there is a chance to insert the endoscope into the hollow organ in order to check for leakage (leakage of the anastomotic part) in the bypass procedure of the hollow organ, the marking is performed at that time. Thereby, when the through-hole is formed early after the procedure, the marking can be used as a guide for the adhered part.

What is claimed is:

1. A tissue treatment method combined with a bypass procedure of a hollow organ, comprising:
a first process of dividing the hollow organ into a first region of a distal side of the hollow organ and a second region of a proximal side of the hollow organ;
a second process of forming a damaged part of a predetermined size on an outer surface of at least one of the first and second regions; and
a third process of fixing the first and second regions such that the damaged part is kept in close contact with an outer surface of the other of the first and second regions.

2. The tissue treatment method according to claim 1, wherein the damaged part is formed in the second process by exposing collagen to the damaged part.

3. The tissue treatment method according to claim 1, wherein the bypass procedure is a Roux-en-Y gastric bypass.

4. The tissue treatment method according to claim 3, wherein the first region is a pouch, and the second region is a remaining stomach.

5. The tissue treatment method according to claim 1, further comprising:
a fourth process of forming a through-hole communicating with the first and second regions with each other by incising an adhered part at which the damaged part and the outer surface of the other are adhered after the third process;
a fifth process of inserting an endoscope into the through-hole and performing a treatment; and
a sixth process of closing the through-hole after the fifth process is completed.
